(19) United States
(12) Patent Application Publication
(10) Pub. No.: US 2022/0287557 A1
(43) Pub. Date: Sep. 15, 2022

(54) METHOD AND DEVICE FOR RETINAL IMAGING BY OPTICAL COHERENCE TOMOGRAPHY

(71) Applicant: Imagine Eyes, Orsay (FR)

(72) Inventors: Nicolas Lefaudeux, Forges-les-Bains (FR); Xavier Levecq, Gif Sur Yvette (FR)

(73) Assignee: Imagine Eyes, Orsay (FR)

(21) Appl. No.: 17/634,784

(22) PCT Filed: Aug. 12, 2020
(86) PCT No.: PCT/EP2020/072656

§ 371 Date: Feb. 11, 2022

(30) Foreign Application Priority Data
Aug. 12, 2019 (FR) FR1909169

(51) Int. Cl. A61B 3/00; A61B 3/10; A61B 3/113; A61B 3/12

(52) U.S. Cl. CPC A61B 3/0008; A61B 3/102; A61B 3/113; A61B 3/12

(57) ABSTRACT

The present description may include a retinal-imaging device, comprising a first module for acquiring tomographic images, with a first illumination and detection sub-module and a first scanning sub-module for scanning in two directions, said first module being configured to acquire a plurality of cross-sectional images of the retina; the device further comprises a second module for acquiring surface images of the retina, with a second illumination and detection sub-module, said second module being configured to acquire surface images of the retina; the device further comprises a control unit configured to determine an angular velocity of the movements of the retina in at least one of the two directions; and to determine, before the start of acquisition of each cross-sectional image of said plurality of cross-sectional images of the retina, a scanning velocity to be applied by said first scanning sub-module in said at least one direction.

### TECHNICAL FIELD OF THE INVENTION

The present description relates to a retinal-imaging method and to a device suitable for implementing said method. More precisely, the retinal-imaging method is based on optical coherence tomography (OCT).

### PRIOR ART

OCT is based on the use of a low-coherence interferometer. This imaging technique allows, in vivo, cross-sectional images of tissues to be taken with an axial resolution of a few microns. One advantage of OCT in ophthalmology stems from its capacity to reveal, in vivo, tissues through other scattering tissues.

FIG. 1A illustrates, in a simplified manner, the main elements of an OCT retinal-imaging device 100 known from the prior art and described, for example, in the review article by J. F. De Boer et al. [Ref. 1]. Such a device for example comprises an illumination and detection module 110 and a scanning module 120 for scanning in two dimensions, which scans a light beam emitted by the module 110 and a beam re-emitted by the retina after illumination by said light beam. The module 110 comprises an illumination sub-module comprising a low-temporal-coherence light source 111, an SLED for example, configured to illuminate a point of the retina with a low-coherence illumination beam. The module 110 moreover comprises a detection sub-module formed from an interferometer, for example a fiber-optic interferometer, for example a Michelson fiber-optic interferometer, comprising a fiber-optic reference arm with a reflecting element 115 and a lens 114. A coupler 113 receives the beams delivered by the fibers 112-1, 112-2, 112-3, which come from the source, the reference arm and the retina, respectively, in order to form interference patterns on a detector 116, a photomultiplier or an avalanche photodiode for example, that is connected to the coupler by a fiber 112-4. The device 100 moreover comprises a signal-processing unit 130, itself connected to a screen and/or interface 140 for a user 11.

Such an optical-coherence-tomography device allows an axial depth image to be obtained at a measurement point on the retina, this axial image, which is acquired in one direction, being called an A-Scan. Such an axial depth image is obtained by various methods as described in [Ref. 1]. For example, an axial depth image may be obtained by means of a source with a broad spectrum and by recording interferograms as a function of wavelength by means of a spectrometer (reference is then made to Fourier-Domain OCT or FD-OCT). According to another example, an axial depth image may be obtained by means of a swept source and by recording an interferogram modulation as a function of time during the sweep of the spectrum of the source (reference is then made to Swept Source OCT or SS-OCT).

Schematic 101 in FIG. 1B thus illustrates an A-Scan of the retina 12 of an eye 10, the axial image being in this example acquired along a propagation axis (S) of the illuminating light beam that is coincident with the axis (A) of the eye, where (A) is defined by the axis passing through the center of the cornea 14 and the center of the fovea.

Moreover, by scanning the light beam in such a way as to move the impact of the beam over the surface of the retina, it is possible to acquire a plurality of axial depth profiles along a line and thus to obtain a two-dimensional cross-sectional image of the retina, this cross-sectional image being called a B-Scan. Schematic 102 in FIG. 1B thus illustrates a B-Scan or cross-sectional image of the retina, acquired in a plane comprising in this example the axis (Δ) of the eye, and with an angular movement θₓ of the scanning beam.

As illustrated in schematic 103 in FIG. 1B, it is also possible to acquire a plurality of cross-sectional tomographic images, which have been referenced B-Scan(i) in schematic 103. The B-scans may be acquired at the same location on the retina, to obtain, by averaging the captured tomographic images, a tomographic image with a better signal-to-noise ratio. The B-scans may also be acquired in various locations on the retina, to obtain a volume image (3D-Scan) of the retina.

Furthermore, as described in published patent application WO 2018197288 [Ref. 2] in the name of the applicant, a multi-scale retinal-imaging system is known, this system having a “wide-field” optical channel for acquiring wide-field images of the retina and a “narrow-field” optical channel for acquiring narrow-field images of high lateral resolution, i.e. typically of a few microns, the narrow-field channel incorporating a wavefront-correcting device. In the aforementioned patent application, imaging may be by OCT.

In practice, although with the OCT devices known to date increasingly high A-Scan acquisition frequencies are being reached (typically higher than 100 kHz), the minimum acquisition time of a cross-sectional image (B-Scan) of the retina, which comprises between 500 and 1000 A-scans, is typically comprised between 3 ms and 10 ms. The minimum acquisition time of a 3D-Scan, which comprises between 200 and 500 B-scans, is typically comprised between 0.5 s and 4 s. However, during such time, the eye may move during image capture and decrease the quality of the acquired images.

Specifically, even when the patient is asked to fixate on a point, the eyes continue to make various types of movement. For example, drift, tremor and involuntary microsaccades are known. In particular, drift is observed as a slow and irregular movement of the optical axes of the eye. Drift, measured as an angular variation in the optical axis of the eye, may reach 150′/s (minutes of arc per second) in a healthy subject. Tremor is a difficult movement to observe since it is an incessant movement, of very low amplitude (20 to 40 seconds of angle) but of high frequency (70 to 90 Hz) and microsaccades, for their part, are minuscule saccades. They may have a minimum dimension of 2-5 minutes of angle and occur involuntarily.

Thus, as illustrated in FIG. 1C, during the acquisition of a B-Scan captured in 10 ms, the ocular axis (Δ) may rotate by almost one minute of arc (schematics 104, 105) with respect to the nominal position (schematic 104) if a subject exhibits a drift of 100′/s, this corresponding, on the retina, to a movement of the illumination beam of about 5 μm. As a result the precision of the obtained image is adversely affected, in particular when an image of high lateral resolution, the resolution of which is able to reach 2 μm, is being acquired. US patent application 2011/0134392 [Ref. 3] describes a high-resolution retinal-imaging method that allows the experimental conditions of acquisition of B-Scans (and in particular the number of A-Scans) to be adapted depending on statistical measurements of eye movements. The described method in particular allows parameters optimized for averaging or oversampling to be obtained, the parameters taking these statistical measurements into account in order to avoid distortion during the acquisition of the B-Scans. However, a stabilizing method that allows eye movements during the acquisition of the B-Scans to be corrected for is not described.

US patent application 2010/0053553 [Ref. 4] describes a retinal-imaging method that allows, during the acquisition of a cross-sectional image (B-Scan) of the retina, eye movements to be tracked and corrected for. More precisely, the method described in the aforementioned document comprises measuring surface images of the retina by means of a first fast acquisition device, for example a laser scanning system. In contrast to the cross-sectional images, the surface images are acquired in a plane perpendicular to the ocular axis. Since the acquisition of the surface images is fast, it allows eye movements to be measured in real time. The method then comprises, in order to acquire a B-Scan, selecting, in the surface image thus acquired, a location and a direction of a desired cross-sectional image, then capturing the B-Scan by means of an OCT device, in which the scanning module is corrected in real time for eye movements measured by the fast acquisition device. The stabilization of the scanning module of the OCT device allows the B-Scan to be captured in the exact location selected in the surface image.

The stabilization of the scanning module of the OCT device via the correction signal delivered by the device for acquiring surface images such as described in [Ref. 4] is ideal in the sense that it allows a B-Scan to be captured with an excellent precision. However, this stabilizing method is complex to implement because it requires a correction signal to be sent a plurality of times during the acquisition of a B-Scan.

Patent application US 2014/0211155 [Ref. 5] also describes a retinal-imaging method that implements stabilization of the OCT measurement by means of a device for fast acquisition of surface images of the retina. More precisely, the method comprises measuring eye movements by means of the acquisition of surface images. The method also comprises acquiring a plurality of cross-sectional tomographic images or B-Scans by means of an OCT device equipped with a module for scanning in two dimensions. In the described method, before each new B-Scan is acquired, it is determined whether the eye movements measured during the acquisition of a previous B-Scan are less than a predetermined threshold value. If this is the case, a correction is made for the eye movements by sending a correction signal to the scanning module of the OCT device before the new B-Scan is acquired, with the aim of correcting the start position. If the eye movements are greater than the predetermined threshold value, the previous B-Scan is acquired again.

The method described in [Ref. 5] is much simpler and faster to implement than the method described in [Ref. 4]. However, during acquisition of a B-Scan, the effect of the drift is observed: drift results, at the end of acquisition, in there being a distance between the theoretical point that it was sought to image and the actual point imaged, this distance possibly in practice being of the order of 5 μm in a healthy subject and much more in subjects who are able to fixate but poorly. Thus, the method described in [Ref. 5] does not allow sufficient stabilization, in particular in the case of imaging of high lateral resolution. One subject of the present description is to provide a retinal-imaging method and device with an acquisition simplicity and speed comparable to that described in [Ref. 5] but which have a precision compatible in particular with imaging of high lateral resolution.

### SUMMARY OF THE INVENTION

According to a first aspect, the present description relates to a retinal-imaging method, comprising:
successively acquiring a plurality of cross-sectional images of the retina by means of a first module for acquiring tomographic images, said first module comprising a first illumination and detection sub-module and a first scanning sub-module for scanning in two directions;
acquiring surface images of the retina by means of a second module for acquiring surface images of the retina, said second module comprising a second illumination and detection sub-module;
determining, on the basis of surface images acquired by the second module, an angular velocity of eye movements in at least one of said directions;
determining, during initiation of acquisition of each cross-sectional image of said plurality of cross-sectional images of the retina, a scanning velocity to be applied by said first scanning sub-module in said at least one direction, said scanning velocity comprising a nominal scanning velocity corrected by a correction velocity depending on said angular velocity of the eye movements.

By no longer systematically correcting the initial position employed for the acquisition of a B-Scan, but rather correcting the scanning velocity with which acquisition of the B-Scan is initiated, depending on the angular velocity of eye movements (or movements of the retina), the applicant has shown that it is possible to much better take into account drift of the retina, in particular in the case of imaging of high lateral resolution, while keeping a system that is simple to implement.

In the present description, the following expressions will be employed interchangeably: lateral resolution and velocity of movement of the retina on the one hand, and angular resolution and angular velocity of the retina such as it appears seen through the anterior segment of the eye on the other hand. Specifically, these lateral and angular values are related via the focal length of the eye. Thus, a resolution of 2 μm is equivalent to an angular resolution of 0.007°, i.e. 0.4 arcmin (with the average focal length of the eye being 17 mm).

A scanning sub-module within the meaning of the present description is configured to scan a light beam emitted by the illumination and detection sub-module, continuously or discretely.

A distinction is drawn, in a known way, between raster or unidirectional scanning, which is always carried out in the same direction, and bidirectional scanning, which is carried out alternately in opposite directions. In both cases, the scanning has a main scanning direction, which is called according to convention the “horizontal direction” in the present patent application.

According to one or more examples of embodiment, the scanning sub-module comprises a resonant or galvanometric scanner configured to deflect a light beam continuously by means of a mirror that is able to rotate about an axis. The scanning sub-module may also comprise a pair of scanners that allow the point of impact of the light beam on the retina to be modified in two directions and that generate a bidirectional scan.

According to one or more examples of embodiment, the scanning sub-module comprises a MEMS mirror (MEMS being the acronym of microelectromechanical system) configured to deflect a light beam in two directions directly.

Generally, any component capable of producing a sufficient number of directions or of points of impact of a light beam on the retina may be used by way of scanning sub-module.

According to one or more examples of embodiment, the angular velocity of the eye movements in a direction is measured on the basis of two surface images of the retina, for example two images acquired successively; the angular velocity is then determined from the angular movement in this direction divided by the time interval separating the acquisitions of said surface images.

According to one or more examples of embodiment, the angular velocity of the eye movements is determined only in one direction, generally the main scanning direction. The correction velocity will then be determined only in this direction and the scanning velocity applied in the other direction by the scanning sub-module will be the nominal velocity.

According to one or more examples of embodiment, the angular velocity of the eye movements will possibly be determined in two directions. In this case, it will be possible to determine a correction velocity in each of the directions. According to one or more examples of embodiment, the scanning velocity applied by the scanning sub-module is, in each direction, a nominal velocity corrected by said correction velocity.

According to one or more examples of embodiment, the correction velocity is a velocity of eye movements measured before the initiation of the acquisition, for example the last velocity of eye movements measured before the initiation of the acquisition.

According to one or more examples of embodiment, the correction velocity is obtained based on a prediction, made on the basis of a history of the velocities of eye movements measured before the initiation of the acquisition.

In the present description, the “nominal scanning velocity” is the predetermined scanning velocity with which a B-Scan is acquired, before correction. The nominal scanning velocity is determined for each of the directions, for example via the total field scanned in said direction, the number of A-scans contained in a B-scan, and the duration of an A-scan; it is generally comprised (values measured in the space of the eye) between ˜100°/s (case of a narrow-field image of high lateral resolution), and more than 10 000°/s (wide-field image). According to one or more examples of embodiment, an angular velocity of the eye movements is determined with a frequency identical to the frequency of acquisition of the surface images. It is also possible to determine the angular velocity with a lower frequency; in practice, between 2 and 10 values of the angular velocity of eye movements will possibly be measured in each direction during a B-Scan.

According to one or more examples of embodiment, the surface images of the retina are acquired by means of a two-dimensional detector of camera type and the acquisition frequency is the acquisition frequency of the camera.

According to one or more examples of embodiment, the surface images of the retina are acquired by scanning a light beam over the retina and the acquisition frequency is dependent on the scanning frequency and on the number of lines in the acquired surface images of the retina.

According to one or more examples of embodiment, the retinal-imaging method further comprises, before each cross-sectional image of said plurality of cross-sectional images of the retina is acquired, determining a shift correction to be applied, by said first scanning sub-module, in at least one of said directions, to a nominal position shift.

In the present description, the “nominal position shift” is a predetermined shift value to be applied, by the first scanning sub-module, before a new B-Scan is acquired, independently of any correction of the movements of the retina. The nominal position shift is for example computed with scanning parameters defined by the user.

According to one or more examples of embodiment, the shift correction to be applied in said at least one direction is dependent on said correction velocity. In particular in the case of a raster scan, it will thus be possible to correct for drift of the retina during the time taken to return to the starting point, for acquisition of a new B-Scan.

According to one or more examples of embodiment, the retinal-imaging method further comprises:
determining, during the acquisition of each cross-sectional image of said plurality of cross-sectional images of the retina, a value representative of a variation in the velocity of the eye movements;
applying, before a subsequent cross-sectional image of the retina is acquired, in said at least one direction, said shift correction to the nominal position shift, if said value representative of the variation in velocity during the acquisition of the previous cross-sectional image, is higher than a predetermined threshold value.

According to one or more examples of embodiment, said value representative of the variation in the velocity of the eye movements corresponds to an angular acceleration of the eye movements (velocity variation during a time interval).

Generally, when the surface images of the retina are acquired by means of a two-dimensional detector of camera type or of a beam-scanning system, said value representative of the variation in the velocity of the eye movements may be determined on the basis of the variation in the velocity of the image of the retina.

According to one or more examples of embodiment, the threshold value is determined depending on the lateral resolution sought for the retinal imaging, the focal length of the eye, the time taken to acquire an A-Scan and the number of A-scans per B-scan. Examples of threshold values are comprised between 0.1°/s and 10°/s.

According to one or more examples of embodiment, the shift correction to be applied, in each of the two directions, if the value representative of the variation in velocity during the acquisition of the previous cross-sectional image, is higher than the predetermined threshold value, is directly computed on the basis of the surface images of the retina.

In other examples, the shift correction may be determined on the basis of one or more previously measured angular velocities of movements of the retina.

Thus, according to one or more examples of embodiment, no correction directly computed on the basis of the surface images of the retina is applied, to the nominal position shift, for the acquisition of a subsequent B-Scan, if there has not been a significant change in the velocity of the movements of the retina. This absence of correction of the position shift, associated with a correction of scanning velocity, allows any abrupt variation in the spacing between the end of one B-scan and the start of the following B-scan to be avoided. A correction, to the nominal position shift, depending on the velocity of any eye movements may however be applied.

According to one or more examples of embodiment, the nominal position shift is predetermined so that a cross-sectional image of the retina is acquired at the same location as that of the previous cross-sectional image of the retina, in order to acquire an averaged cross-sectional image of the retina. The imaging method then comprises determining an averaged cross-sectional image of the retina, computed on the basis of an average of a plurality of cross-sectional images acquired at the same location. According to one or more examples of embodiment, said averaged cross-sectional image of the retina is displayed. According to one or more examples of embodiment, the nominal position shift is predetermined so that a cross-sectional image of the retina is acquired at the same location as that of the previous cross-sectional image of the retina, in order to acquire a so-called “angiographic” image of the retina, or “OCT-A” image, allowing the vessels of the retina (see [Ref. 1]) to be highlighted; the method then comprises determining an image computed on the basis of a modification of content between a plurality of cross-sectional images acquired at the same location. For example, an indicator of dispersion as regards the information of each pixel of the image may be used. The information of each pixel may for example correspond to a modulus of a complex value corresponding to the OCT measurement, or to the phase of this measurement. An indicator of dispersion is for example the variance, the standard deviation, the mean absolute deviation, the moment of order N or any other indicator of dispersion of the information of each pixel.

According to one or more examples of embodiment, the nominal position shift is predetermined so that a cross-sectional image of the retina is acquired in a location on the retina that is different from that of the previous cross-sectional image, in order to acquire a three-dimensional image of the retina.

According to one or more examples of embodiment, the method further comprises displaying a three-dimensional image of the retina, formed on the basis of said plurality of cross-sectional images of the retina.

On the basis of said three-dimensional image of the retina, obtained by means of the method according to the first aspect, a user may, according to one or more examples of embodiment, select a region of interest to be imaged. He may for example, and non-limitingly, require an averaged B-Scan to be acquired, a three-dimensional image of the retina to be acquired with high lateral resolution in a narrow field, or an angiographic image of the retina to be acquired.

To each of these applications, the method according to the present description may be applied, in particular with a correction of the scanning velocity at the start of acquisition of a B-Scan and, if necessary, a correction of shift.

According to one or more examples of embodiment, the method further comprises, during the acquisition of each cross-sectional image of the retina, detecting an eye blink on the basis of said surface images of the retina. During an eye blink, the obtained surface images of the retina exhibit an almost zero signal.

In the event of detection of an eye blink, acquisition of the current cross-sectional image of the retina is stopped, then, when the blink has ended and the surface images of the retina are once more being obtained, a position shift is applied, in each of the directions, by said first scanning sub-module, to restart acquisition of said cross-sectional image of the retina.

According to one or more examples of embodiment, the method further comprises, during the acquisition of each cross-sectional image of the retina, detecting a microsaccade on the basis of said angular velocity of the movements of the retina. In the event of detection of a microsaccade, acquisition of the current cross-sectional image of the retina is stopped, and, when the microsaccade has ended, a position shift is applied, in each of the directions, by said first scanning sub-module, to restart acquisition of said cross-sectional image of the retina.

Specifically, the applicant has shown that, in the event of a blink, the absence of signal allows no measurement, and, in the event of a microsaccade, the movement of the retina during the acquisition is too large to be able to be corrected. It is then preferable to restart acquisition of the B-scan.

According to a second aspect, the present description relates to devices for implementing described methods according to one or more embodiments of the method according to the first aspect.

Thus, the present description relates to a retinal-imaging device, comprising:
a first module for acquiring tomographic images, comprising a first illumination and detection sub-module and a first scanning sub-module for scanning in two directions, said first module being configured to acquire a plurality of cross-sectional images of the retina;
a second module for acquiring surface images of the retina, comprising a second illumination and detection sub-module;
a control unit configured to
   determine, on the basis of surface images acquired by the second module, an angular velocity of eye movements in at least one of said directions;
   determine, during initiation of acquisition of each cross-sectional image of said plurality of cross-sectional images of the retina, a scanning velocity to be applied by said first scanning sub-module in said at least one direction, said scanning velocity comprising a nominal scanning velocity corrected by a correction velocity depending on said angular velocity of the eye movements.

According to one or more examples of embodiment, each of said first and second modules comprises a wide-field optical channel and a narrow-field optical channel. Such modules are described for example in [Ref. 2].

According to one or more examples of embodiment, said second module for acquiring surface images of the retina further comprises a second scanning sub-module for scanning in two directions. It is for example a question of a scanning laser ophthalmoscope (SLO). Such an SLO for example comprises a resonant scanner for scanning in a first direction and a galvanometric scanner for scanning in a second direction.

According to one or more examples of embodiment, said first module for acquiring tomographic images is a FD OCT or a SS OCT module such as known from the prior art.

### BRIEF DESCRIPTION OF THE FIGURES

Other advantages and features of the invention will become apparent on reading the description, which is illustrated by the following figures:

FIG. 1A, which has already been described, shows a schematic illustrating an OCT retinal-imaging device known from the prior art.

FIG. 1B, which has already been described, shows three schematics illustrating (Δ-Scan and B-Scan) OCT retinal images known from the prior art;

FIG. 1C, which has already been described, shows two schematics illustrating the effect of drift of the eye on (B-Scan) OCT retinal images;

FIG. 2 shows a schematic illustrating an example of an optical-coherence-tomography (OCT) retinal-imaging device according to the present description;

FIG. 3A shows a block schematic illustrating an example of an optical-coherence-tomography (OCT) retinal-imaging method according to the present description;

FIG. 3B shows a block schematic illustrating a detail of the retinal-imaging method illustrated in FIG. 3A, according to one example;

FIG. 4A shows a schematic illustrating two curves showing, as a function of time and in one direction, an example of drift of the retina and the corresponding correction signal in one example of a retinal-imaging method according to the present description, respectively;

FIG. 4B shows, by way of comparison, a schematic illustrating the same two curves as in FIG. 4A but in an example of a retinal-imaging method according to [Ref. 4];

FIG. 4C shows, by way of comparison, a schematic illustrating the same two curves as in FIG. 4A but in an example of a retinal-imaging method according to [Ref. 5];

FIG. 5 shows a block schematic of an example of an optical-coherence-tomography (OCT) retinal-imaging method according to the present description, comprising acquiring a 3D OCT image of the retina, and a user selecting other images to be acquired on the basis of a display of the 3D image.

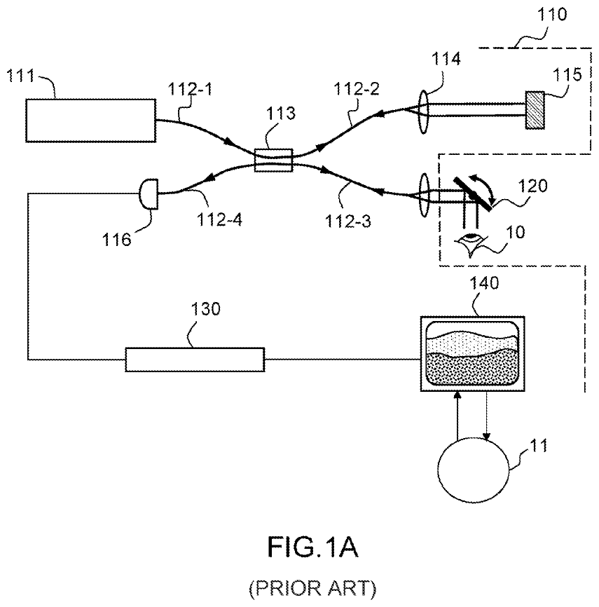

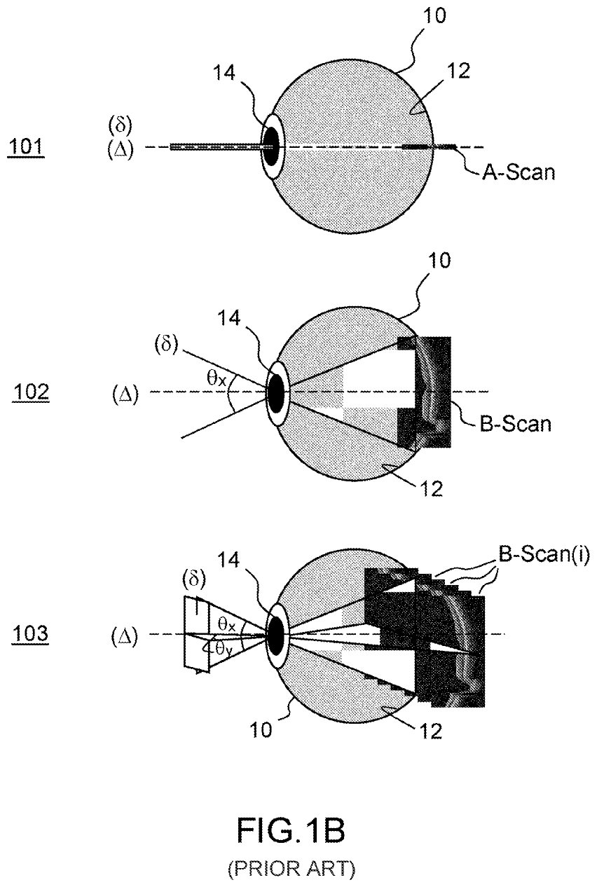

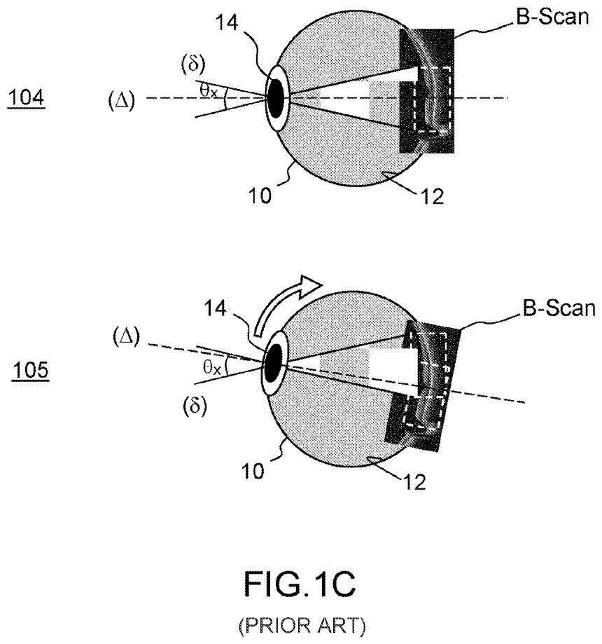

### DETAILED DESCRIPTION OF THE INVENTION

In the figures, the elements have not been shown to scale for better legibility.

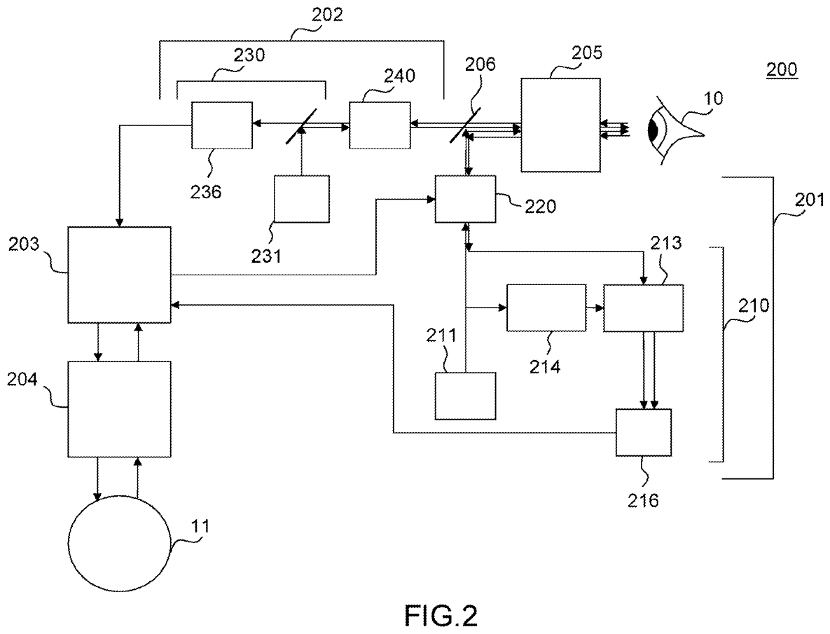

FIG. 2 shows a schematic illustrating one example of an optical-coherence-tomography (OCT) retinal-imaging device 200 configured to implement examples of retinal-imaging methods according to the present description.

The device 200 comprises a first module 201 for acquiring tomographic images, a second module 202 for acquiring surface images of the retina and a control unit 203 for controlling elements of said first and second modules 201, 202 and for carrying out imaging-method steps according to the present description. A beam-splitting element 206 allows the surface-imaging and tomography channels to be combined for illumination purposes, and said channels to be split for detection purposes.

Generally, the control unit to which reference is made in the present description may comprise one or more physical entities, for example one or more computers. When, in the present description, reference is made to steps of computing or processing in particular in order to carry out steps of a method, it will be understood that each computing or processing step may be implemented by software, hardware, firmware, microcode, or any suitable combination of these technologies. When software is used, each computing or processing step may be carried out by computer-program instructions or software code. These instructions may be stored or transmitted to a storage medium that is readable by the control unit and/or be executed by the control unit in order to carry out these computing or processing steps.

The control unit 203 is, in this example, connected to a screen and/or interface 204 in order to interface with a user 11.

The first module 201 for acquiring tomographic images comprises, in a known way, a first illumination and detection sub-module 210 and a first scanning sub-module 220 for scanning, in two directions, a light beam emitted by the sub-module 210 and a beam re-emitted by the retina after illumination by said light beam.

The illumination and detection sub-module 210 comprises a low-temporal-coherence light source 211, an SLED for example, configured to illuminate a point on the retina with a low-coherence illumination beam, and an interferometer 213, for example a fiber-optic interferometer, for example a Michelson fiber-optic interferometer, comprising a reference arm 214, and which is configured to form interference patterns on a detector 216, a photomultiplier or an avalanche photodiode for example. The scanning sub-module 220 for example comprises two galvanometric scanners. The elements of the first module 201 for acquiring tomographic images are known and configured to generate tomographic images of FD-OCT or SS-OCT type, such as described for example in [Ref. 1]. Only the main elements are schematically shown in FIG. 2.

The second module 202 for acquiring surface images of the retina comprises, in the example of FIG. 2, a second illumination and detection sub-module 230 and a second scanning sub-module 240 for scanning, in two directions, a light beam emitted by the sub-module 230 and a beam re-emitted by the retina after illumination by said light beam. The second module 202 is for example a scanning laser ophthalmoscope (SLO) known from the prior art, such as described in R. H. Webb et al. [Ref. 6]. The illumination and detection sub-module 230 comprises a source 231, an infrared superluminescent diode (SLED) for example, and a detector 236, an avalanche photodiode for example. The scanning sub-module 240 comprises a resonant scanner and a galvanometric scanner.

The second module 202 for acquiring surface images of the retina thus allows surface images of the retina to be acquired at a given acquisition frequency, which is determined by the scanning frequency of the scanning sub-module 240 and the number of points per line of the surface images of the retina that it is sought to acquire.

It will be noted that other modules for acquiring surface images of the retina may be used, such as for example a two-dimensional acquisition device of camera type. In this case, the acquisition frequency is given by the acquisition frequency of the camera. Generally, an SLO, although more complex to implement, yields images that are more contrasted than those provided by a camera.

In the example of FIG. 2, an optical system allows, on each of the retina-surface-imaging and tomography channels, the light beam to be conveyed to the patient's eye and it to be shaped conjointly with the scanning module for scanning the retina in the desired way. The optical systems of each of the channels have at least one common element 205.

It will be noted that, according to one or more examples of embodiment, each of said first and second modules may comprise a wide-field optical channel and a narrow-field optical channel (which have not been shown in FIG. 2). Such modules are described for example in [Ref. 2].

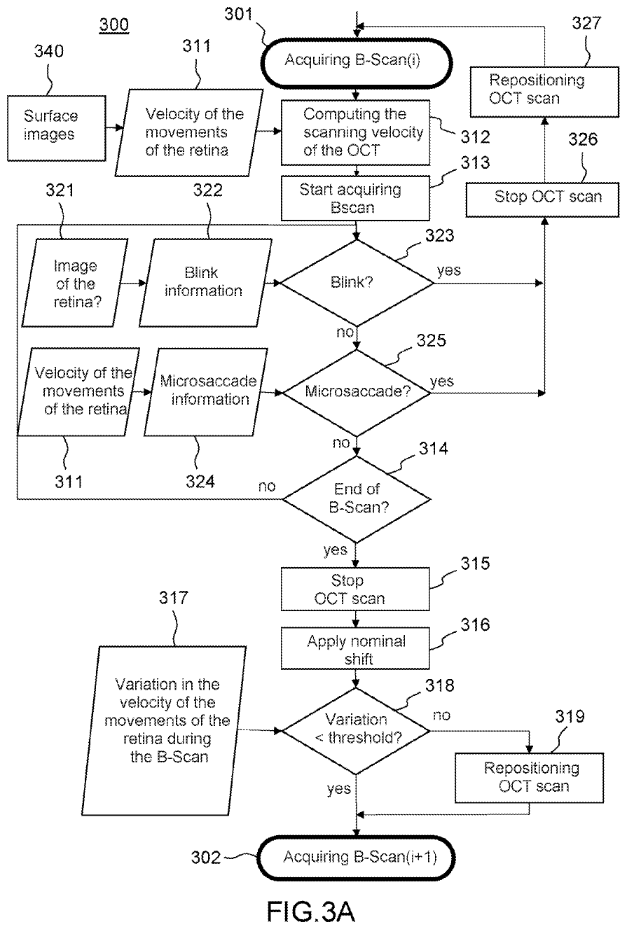

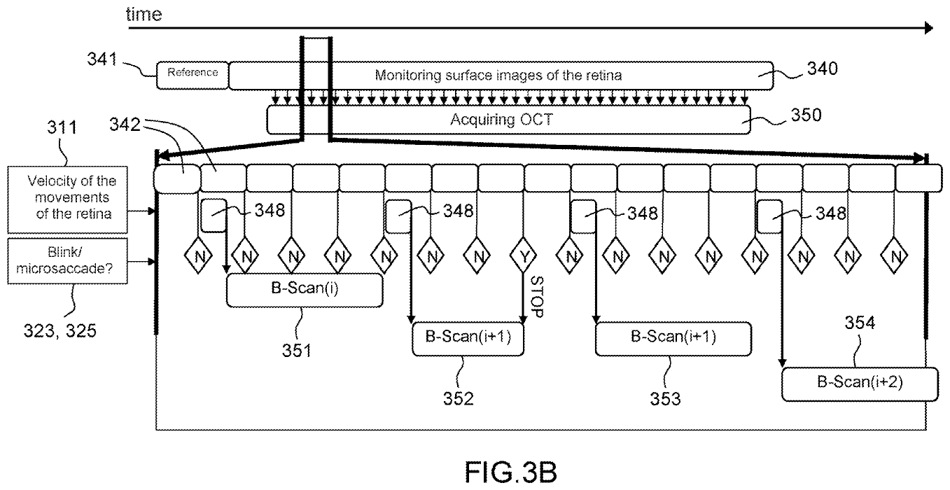

FIG. 3A shows a block schematic of one example of an optical-coherence-tomography (OCT) retinal-imaging method 300 according to the present description, which method is implemented, for example, by means of a device as illustrated in FIG. 2, and FIG. 3B shows a block schematic illustrating a detail of the retinal-imaging method illustrated in FIG. 3A. In the method according to the present description, the surface images of the retina are acquired at a given frequency by the module (202, FIG. 2) for acquiring (step 340, FIG. 3B) surface images. In practice, a series of surface images of the retina are acquired to generate a reference image 341 via registration and averaging, then the acquired images are compared to identify the presence of a blink, of a microsaccade and to measure the velocity of the movements of the retina.

On the basis of the surface images of the retina, the control unit determines (step 311) an angular velocity of the eye movements (or movements of the retina) in the two scanning directions, or in at least one of the two directions, the main direction for example. The angular velocity of the movements of the retina may be determined at the acquisition frequency of the surface images, and for example on the basis of successively acquired surface images.

The retinal-imaging method 300 moreover comprises successively acquiring (350, FIG. 3B) a plurality of cross-sectional images B-Scan(i) of the retina by means of the first module (201, FIG. 2) for acquiring tomographic images.

As illustrated in FIG. 3A, during the acquisition 301 of each cross-sectional image B-Scan(i), a scanning velocity 312 is computed by the control unit 203. The scanning velocity is applied by the first scanning sub-module 220. The scanning velocity to be applied before the start of acquisition 313 of B-Scan(i) comprises a nominal scanning velocity corrected by a correction velocity depending on the angular velocity of the eye movements before initiation of the acquisition of the image B-Scan(i). The last value of the angular velocity of the eye movements measured before the initiation of the acquisition of the B-Scan will be taken by way of example. It is also possible to determine a correction velocity on the basis of a prediction made on the basis of a plurality of previously determined velocity values of the movements of the retina.

During the acquisition of a B-Scan, it is determined (step 322) whether an eye blink has occurred on the basis of the surface images of the retina. To do this, an absence of signal is detected.

It is also determined (step 324) whether a microsaccade has occurred. The information regarding a microsaccade is determined on the basis of the velocities of the movements of the retina. For example, a very rapid shift in the image will possibly be observed.

If a blink (323) or microsaccade (325) occurs, the acquisition of the B-Scan is suspended (326) for the duration of the blink or of the microsaccade. A completely new acquisition of the current cross-sectional image is performed after the end of the eye blink or of the microsaccade. To do this, a shift is applied to the scanning beam (327) and a new measurement of the velocity of movement of the retina is taken into account.

If no microsaccade occurs, the acquisition of the B-Scan continues until the end of the line determined beforehand for acquisition has been reached (314).

The OCT scan stops (315) and a new B-Scan is started if necessary.

In order to start a new B-Scan, a nominal shift is applied (316). The nominal position shift is a predetermined shift value to be applied, by the first scanning sub-module, before a new B-Scan is acquired, independently of any correction of eye movement. The nominal position shift is for example computed with scanning parameters defined by the user. For example, the user defines a raster scan (scan always in the same direction) that yields B-scans that are 30° in length and spaced apart vertically by 0.2°. The nominal position shift between the end of a B-scan and the following B-scan is for example −30°, −0.2°.

In the case of a raster scan, it will be possible to apply, to the nominal shift, a shift correction in at least one direction, the main direction for example, the shift correction being dependent on the angular velocity of the eye movements in said direction and on the time taken by the scanner to return to the starting point. This makes it possible to correct for drift of the retina during the time taken to return to the starting point, for acquisition of a new B-Scan.

In the example illustrated in FIG. 3A, a variation in the velocity of the movements of the retina is also determined (317) during the acquisition of the B-Scan(i). This measurement of the variation in velocity is used to determine whether or not to apply a correction shift to be applied before the acquisition of a subsequent B-Scan.

Thus, it is determined (318) whether the variation is smaller than a predetermined threshold. If so, the following B-Scan (B-Scan(i+1)) is acquired (302) without shift correction (or only with the velocity-related correction explained above). If not, a shift correction is applied (319) before acquiring (302) the following B-Scan (B-Scan(i+1)). This shift correction is computed based on the surface images of the retina and is applied in at least one of the scanning directions.

Thus, in practice, when a plurality of cross-sectional images of the retina are acquired, during acquisition of each of the cross-sectional images, a scanning velocity comprising a nominal scanning velocity corrected for the velocity of the movements of the retina is applied by the first scanning sub-module. Moreover, before initiation of each new cross-sectional image of the retina, a shift correction in at least one direction may be applied to the nominal position shift by said first scanning sub-module. This shift correction may be a correction computed directly on the basis of the images of the retina if said value representative of the variation in the velocity of the movements of the retina during the acquisition of the previous cross-sectional image, is higher than the predetermined threshold value.

The threshold value will possibly be determined depending on the lateral resolution sought for the retinal imaging, the focal length of the eye, the time taken to acquire an A-Scan and the number of A-scans per B-scan. Examples of threshold values are comprised between 0.1°/s and 10°/s. These values are computed for (min value) a resolution of 1 μm, an A-Scan acquisition frequency of 50 kHz, and a number of 1000 A-Scans per B-Scan and for (max value) a resolution of 5 μm, an A-Scan acquisition frequency of 200 kHz, and a number of 500 A-Scans per B-Scan.

By way of example, a lateral resolution on the retina of 2 μm with an eye with a focal length of 17 mm corresponds to an angular resolution of 0.007°. The acquisition time of a B-Scan composed of 1000 A-Scans acquired at a frequency of 200 kHz is 5 ms. The velocity-variation threshold to be applied in this case is 0.007°/0.005 s=1.3°/s.

FIG. 3B thus illustrates an example of acquisition of a first B-Scan(i) (step 351). During acquisition, the control unit determines, on the basis of the surface images 342, a velocity of the movements of the retina (step 311). At the end of the acquisition, a new B-Scan is acquired (B-Scan(i+1), step 352). As illustrated in FIG. 3B, the B-Scan(i+1) is interrupted due to detection of a blink or of a microsaccade. It is therefore acquired again (353). Then a new B-Scan is acquired (B-Scan(i+2), 354), etc.

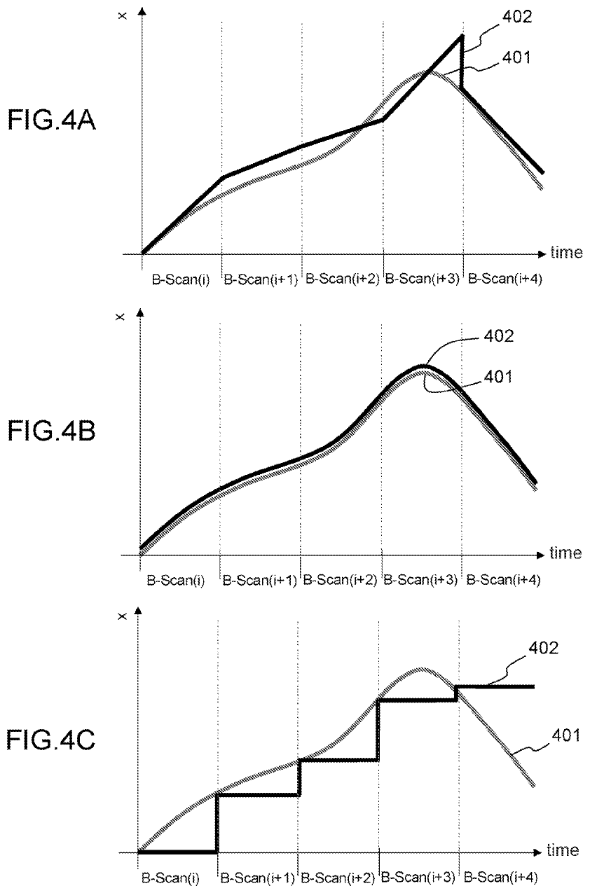

FIG. 4A shows a schematic illustrating two curves 401, 402 showing, as a function of time and in one direction, an example of drift of the retina (401) and the corresponding correction signal (402) applied to the scanning sub-module (220, FIG. 2) of the module for acquiring tomographic images, respectively.

For the acquisition of the cross-sectional images of the retina B-Scan(i), B-Scan(i+1), B-Scan(i+2), B-Scan(i+3), no shift correction is applied but only a correction of scanning velocity, which is observable by the changes in slope. In contrast, during acquisition of the cross-sectional image B-Scan(i+4), the control unit detects a variation in the angular velocity of the movements of the retina larger than the predetermined threshold and a shift correction is applied, in addition to the correction of the scanning velocity.

These curves may be compared with those obtained (see FIG. 4B) in an example of a retinal-imaging method according to [Ref. 4]. In this example, a shift correction is applied with a high frequency during the acquisition of the B-Scan, and hence the two curves follow each other precisely.

The curves in FIG. 4A may also be compared with those obtained (see FIG. 4C) in an example of a retinal-imaging method according to [Ref. 5]. In this example there is no correction of scanning velocity and shift is systematically corrected depending on the surface images of the retina. As may be seen in FIG. 4C, the resulting correction is much less precise.

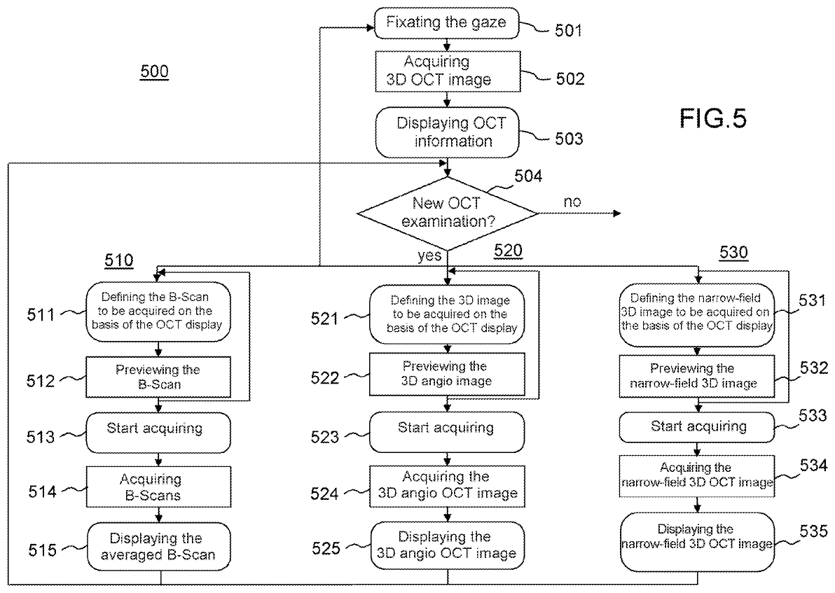

FIG. 5 shows a block schematic of an example of an optical-coherence-tomography (OCT) retinal-imaging method according to the present description, comprising acquiring a 3D OCT image of the retina, and a user selecting other images to be acquired on the basis of a display of the 3D image.

More precisely, in this example, the method 500 comprises a step 501 in which a patient is asked to fixate his gaze on a reference point.

A three-dimensional OCT image of the retina is acquired (502) by means of a method according to the present description—for example a method such as described with reference to FIG. 3A, and in which successive B-Scans are acquired in various locations on the retina. The 3D OCT image is displayed (503) and a user is free to request a new examination (504). Thus, in this example of embodiment, a user may for example select a new region of the retina to be imaged based on the previously acquired three-dimensional image.

FIG. 5 illustrates a plurality of examples of complementary examinations 510, 520, 530 that may be chosen by a user, these examples being non-limiting and being able to be implemented successively based on the same 3D image of the retina.

For example, a user may choose to carry out an averaged B-Scan (510).

The position of the B-Scan to be carried out is chosen (step 511) on the basis of the 3D retinal image determined beforehand and displayed. A preview may be shown (512) before the acquisition (513) is started. A plurality of B-Scans are acquired (514) according to the method according to the present description, for example the method such as described with reference to FIG. 3A. In particular, the nominal position shift is predetermined so that a cross-sectional image of the retina is acquired at the same location as that of the previous cross-sectional image of the retina, and an averaged cross-sectional image of the retina is computed on the basis of an average of a plurality of cross-sectional images acquired at the same location. The averaged B-scan is displayed (515).

According to another example, a user may choose to take (520) a so-called angiographic, three-dimensional image of the retina.

The position of the region to be imaged is chosen (step 521) on the basis of the 3D retinal image determined beforehand and displayed. A preview may be shown (522) before the acquisition (523) is started. A plurality of B-Scans are acquired (524) according to a method according to the present description, for example the method such as described with reference to FIG. 3A. In this example, an image is computed on the basis of a modification of content between a plurality of images acquired at the same location. The angiographic image is displayed (525).

In another example, a user may choose to take (530) a narrow-field three-dimensional retinal image of high lateral resolution.

The position of the region to be imaged is chosen (step 531) on the basis of the 3D retinal image determined beforehand and displayed. A preview may be shown (532) before the acquisition (533) is started. A plurality of B-Scans are acquired (534) according to a method according to the present description, for example the method such as described with reference to FIG. 3A, using the high-resolution narrow-field channels of the retinal-imaging device. The three-dimensional image of the retina is displayed (535).

Although described through a number of examples of embodiment, the retinal-imaging method and device according to the present description comprises various variants, modifications and improvements that will appear obvious to those skilled in the art, it being understood that these various variants, modifications and improvements form part of the scope of the invention such as defined by the following claims.

### REFERENCES

Ref 1.: J. F. De Boer et al. “Twenty-five years of optical coherence tomography: the paradigm shift in sensitivity and speed provided by Fourier domain OCT”, Vol. 8, No. 7 1 Jul. 2017|BIOMEDICAL OPTICS EXPRESS 3248
Ref 2.: WO 2018197288
Ref 3.: US 2011/0134392
Ref 4.: US 2010/0053553
Ref 5.: US 2014/0211155
Ref 6.: R. H. Webb et al. “Confocal scanning laser ophthalmoscope” Appl. Opt. 26, 1492-1499 (1987)

1. A retinal-imaging method, comprising:
successively acquiring a plurality of cross-sectional image (B-Scan(i)) of the retina by means of a first module for acquiring tomographic images, said first module comprising a first illumination and detection sub-module and a first scanning sub-module for scanning in two directions (x, y);
acquiring surface images of the retina by means of a second module for acquiring surface images of the retina, said second module comprising a second illumination and detection sub-module;
determining, on the basis of surface images acquired by the second module, an angular velocity of the movements of the retina in at least one of said directions (x, y);
determining, before the start of acquisition of each cross-sectional image of said plurality of cross-sectional images of the retina, a scanning velocity to be applied by said first scanning sub-module in said at least one direction, said scanning velocity comprising a nominal scanning velocity corrected by a correction velocity depending on said angular velocity of the movements of the retina.

2. The retinal-imaging method as claimed in claim 1, further comprising, before each cross-sectional image of said plurality of cross-sectional images of the retina is acquired, determining a shift correction to be applied to a nominal position shift by said first scanning sub-module, in at least one of said directions.

3. The retinal-imaging method as claimed in claim 2, wherein the shift correction to be applied in said at least one direction is dependent on said determined correction velocity in said at least one direction.

4. The retinal-imaging method as claimed in claim 1, wherein the retinal-imaging method further comprises:
determining, during the acquisition of each cross-sectional image (B-Scan(i)) of said plurality of cross-sectional images of the retina, a value representative of a variation in the velocity of the eye movements in at least one of said directions;
applying, before a subsequent cross-sectional image (B-Scan(i+1)) of the retina is acquired, a shift correction to be applied to a nominal position shift in said at least one direction, if said value representative of the variation in velocity during the acquisition of the previous cross-sectional image (B-Scan(i)), is higher than a predetermined threshold value.

5. The imaging method as claimed in claim 4, wherein said shift correction is directly computed on the basis of the surface images of the retina.

6. The retinal-imaging method as claimed in claim 4, wherein said value representative of the variation in the velocity of the movements of the retina comprises a value of the acceleration of the movements of the retina.

7. The retinal-imaging method as claimed in claim 1, further comprising determining an averaged cross-sectional image of the retina, computed on the basis of an average of a plurality of cross-sectional images acquired at the same location.

8. The retinal-imaging method as claimed in claim 1, further comprising determining an image computed on the basis of an estimation of a modification of content between a plurality of cross-sectional images acquired at the same location.

9. The retinal-imaging method as claimed in claim 1, wherein said plurality of cross-sectional images of the retina are acquired in various locations on the retina, in order to acquire a three-dimensional image of the retina.

10. The retinal-imaging method as claimed in claim 9, further comprising:
displaying said three-dimensional image of the retina;
selecting, by a user, on the basis of said three-dimensional image of the retina, a new region of the retina to be imaged.

11. The retinal-imaging method as claimed in claim 1, further comprising:
detecting, during the acquisition of each cross-sectional image (B-Scan(i)) of said plurality of cross-sectional images of the retina, an eye blink on the basis of said surface images of the retina;
stopping acquiring said cross-sectional image (B-Scan(i)) in the event of detection of an eye blink; and
applying, by means of said first scanning sub-module, a position shift in each of the directions, to restart acquisition of said cross-sectional image of the retina.

12. The retinal-imaging method as claimed in claim 1, further comprising:
detecting, during the acquisition of each cross-sectional image (B-Scan(i)) of said plurality of cross-sectional images of the retina, a microsaccade on the basis of said angular velocity of the movements of the retina,
stopping acquiring said cross-sectional image (B-Scan(i)) in the event of detection of a microsaccade;
applying, by means of said first scanning sub-module, a position shift in each of the directions, to restart acquisition of said cross-sectional image of the retina.

13. A retinal-imaging device, comprising:
a first module for acquiring tomographic images, comprising a first illumination and detection sub-module and a first scanning sub-module for scanning in two directions, said first module being configured to acquire a plurality of cross-sectional images (B-Scan(i)) of the retina;
a second module for acquiring surface images of the retina, comprising a second illumination and detection sub-module, said second module being configured to acquire surface images of the retina;
a control unit configured to:
determine, on the basis of surface images acquired by the second module, an angular velocity of the movements of the retina in at least one of the two directions;
determine, before the start of acquisition of each cross-sectional image of said plurality of cross-sectional images of the retina, a scanning velocity to be applied by said first scanning sub-module in said at least one direction, said scanning velocity comprising a nominal scanning velocity corrected by a correction velocity depending on the angular velocity of the movements of the retina.

14. The retinal-imaging device as claimed in claim 13, wherein each of said first and second modules comprises a wide-field optical channel and a narrow-field optical channel.

15. The retinal-imaging device as claimed in claim 13, wherein said second module for acquiring surface images of the retina further comprises a second scanning sub-module for scanning in two directions.
